(19) United States
(12) Patent Application Publication
(10) Pub. No.: US 2026/0233074 A1
(43) Pub. Date: Aug. 13, 2026

(54) FORCE MEASURING DEVICE FOR HIGH-STRIKER

(71) Applicant: High Striker UK Limited, London Greater London (GB)

(72) Inventor: Jonathan Scott Paulson, London Greater London (GB)

(21) Appl. No.: 19/155,999

(22) PCT Filed: Feb. 13, 2024
(86) PCT No.: PCT/EP2024/053645

§ 371 Date: Aug. 13, 2025

(30) Foreign Application Priority Data
Feb. 13, 2023 (GB) 2302009.2

(51) Int. Cl. A63B 69/32; A63B 24/00; A63B 71/06

(52) U.S. Cl. CPC A63B 69/325; A63B 24/0021; A63B 24/0062; A63B 71/0622; A63B 71/0669; A63B 2024/0043; A63B 2024/0065; A63B 2071/0694; A63B 2220/53; A63B 2220/833

(57) ABSTRACT

A portable measuring device to replace a carnival or fairground Hi-Striker is provided, adapted to be used in the gym or as a piece of gym equipment. The device has a target impact area with concentric, differently coloured rings (like a bullseye) that is to be hit, impacted or struck, with a sledgehammer, mace or slam ball. The device can be located horizontally, on the floor, or vertically, on a wall, and can be securely fixed to either. The device measures force, or pressure, of an impact. It can also measure the location, and so determine accuracy (of each impact or force). The device has a display which can display impact parameters, and therefore act as a performance monitor. It may give the number of impacts, time of impacts, location of impacts. The device may therefore give a score or measurement of force combined with accuracy and location.

### INTRODUCTION

The present invention relates to gym (or gymnasium equipment), in particular what is commonly known as a Hi-Striker (or replacement therefor). The invention is thus a device for measuring impact force, such as a hit or a strike, usually with a slam ball or sledgehammer (or mace or other piece of striking or hitting equipment or equivalent). It is a force or impact measuring device that can be located on the floor, or a wall, and can be easily moved therebetween.

### BACKGROUND

Sledgehammer training is popular amongst fitness enthusiasts. However, while many parameters in functional fitness is measurable (for example, number of push-ups, the weight of the bar lifted overhead, the time to sprint a mile), sledgehammer training (and the parameters associated therewith) is not.

Traditionally, in carnivals and fairs one sees the “Hi-Striker” attraction that measures the force of each user's sledgehammer swing. For decades, a Hi-Striker carnival game involves a player hitting a pad with a (sledge) hammer and the force of the impact is measured mechanically. This is usually shown on a vertical scale, which sometimes has a bell at the top which is rung if a vertical slider is pushed to the top of the scale by the force of the impact. These carnival or fairground machines measure the force mechanically either by air pressure or water displacement.

In other methods, fitness training involves hitting a tyre or half tyre with a sledgehammer, or using a combined sledge and tyre single hammer, or using a weighted block on a track (which is normally positioned between the legs, and with the hammer at foot level).

More recently, various pieces of gym equipment allow or measure kicking, throwing, jumping or hitting. Some include sensors inside a striking pad to measure the force of impact, or measure controlled compression. Others provide a simple platform to measure human vertical jumps, squats, stomps etc. Other devices include sensors inside a medicine ball to measure power, force and speed, or include sensors inside a soccer football to track power, spin, strike and trajectory.

However, many of these devices have limits or problems, and do not solve the idea of being able to accurately measure hitting or impact force. For example, if one is simply hitting a tyre, it is almost impossible to tell how hard the tyre is being hit. One cannot compare two people competing against each other, as there is no way to objectively measure and score the force impacts. In addition, the prior art does nor measure accuracy (of the strike or hit).

The present invention aims to overcome, or at least mitigate, some of the problems and draw backs of these prior art methods.

### DESCRIPTION OF THE INVENTION

According to a first aspect of the invention, there is a provided an (e.g. portable or compact) force and/or impact measuring device or unit, This may be a high-striker, or a strength tester device or apparatus. It can be an (electronic) test-of-strength or high striker machine, or a fitness evaluation or capability device, or a strike assessment apparatus. (There is no bell, (moving or sliding) puck and/or vertical tower or slide). It may be a high striker game apparatus.

This can be a (piece of) gym (or gymnasium) equipment. The device can measure impact and/or force, such as using one or more sensor(s).

The device usually comprises a target and/or impact area. It will usually be designed to be hit, impacted or struck, for example with a (sledge)hammer, mace, mallet, slam ball or equivalent. This, a high impact device. It may absorb (substantially) all of the kinetic energy (from the impact). It may comprise a heavy base so that it does not move on impact.

The device may be able to measure and/or indicate or display the location of the or each impact and/or the concentration (or accuracy) of the force/pressure and/or impact. Thus, the device may have one or more sensor(s) such as to achieve this. The or each sensor can be capable of measuring force (and/or impact) and location (and/or accuracy). The location of impact(s) can give an indication of (the user's) accuracy of hitting or impacting the target (or impact) area.

The device may therefore have one or more force or impact sensor(s). These may be able to measure and/or calculate force and/or impact. The, or each, sensor(s) may measure the or each parameter electrically, electronically or digitally (and not mechanically). This may comprise force sensing capacitance. The device may g=have a display for one or more of these parameters

The device may also have one or more location/impact sensor(s). These may comprise a piezo sensor and/or array. The sensor(s) may be arranged in a square, rectangular, circular or honeycomb fashion or array. The sensors may be electric and/or electronic sensor(s) and suitably have an (electrical) connection to a display/electronics unit. They may be located on, or between, a plate(s). They are not mechanical. The electrical sensors may be passive and/or active.

The target or target area is designed to be impacted, hit or struck. It may therefore be resistant to impact, and/or be able to absorb impact. It may comprise suitably tough and/or rugged material(s). It may comprise a rubber surface. The surface is suitably relatively hard and/or made of a hard material, so that it is substantially impact-resistant. The (target) surface may be non-deformable (for example, it is suitably not a foam or mat but an impacted or impactful surface).

The target area may be substantially circular or another suitable shape (e.g. square or rectangular). It may be provided with one or more or a series of concentric rings. The sensors may be able to detect an impact in one or more of the (concentric) rings. Suitably, the rings (or annuli) are coloured, and suitably differently coloured (from each other), or otherwise differentiable (from each other)

There may be one or more sensor(s) for each ring. For example, there may be sensors for each ring, or the sensors may be able to detect an impact in one or more rings. The target area may therefore resemble a bullseye appearance, for example it may have a central red ring, and outer rings that may be different colours, such as white and/or blue. The device may be able to detect impact in each of the different (coloured) rings. It may be able to detect or measure the force of impact. Suitably the device comprises multiple layers or plates.

The device may be generally or substantially circular or cylindrical in shape, for example resembling a (tree) “stump”. It may comprise multiple and/or layered or stacked (e.g., substantially circular) plates, suitably comprised of different materials.

The plurality or multiplicity of plates may (all) be of a similar size and/or diameter. They may comprise plates, each suitably comprising metal, rubber, polymer, polycarbonate and/or other suitable material. The plates may be sufficiently strong and non-deformable to withstand (multiple) impact(s). However, there may additionally be some, for example limited flexibility, and may comprise an impact absorbing material such as rubber, foam and/or sponge. The material may thus be able absorb kinetic energy (of the impact).

The display may be insulated from the plates or device (e.g. with a vibration proof material and/or shock absorber such as foam, sponge) so as to reduce vibration and/or damage (to the display) from the impacts.

The device may comprise an electronics unit (e.g. computer) which is capable of receiving and/or processing parameters and/or information (such as from the sensors). This may concern the impacts (such as force and/or location). It may be able to communicate or send those parameters or information to the display and/or perform calculations or use an algorithm or equations to calculate scores.

The unit may be integral with the display, or they may be separate and in communication with each other (such as Bluetooth, WiFi, etc). The unit may have a clock/timer so can time impacts (or time gaps between them) and/or process the number of impacts. The unit may be insulated electrically and/or vibrationally from the device (so as to prevent damage to the unit).

The device may thus comprise a display which may be able to display certain impact and/or force parameters. It may therefore comprise a performance monitor. The display suitably comprises an LED and/or LCD display. The display is suitably integral with the device, for example, placed on one side or located or fixed to a (side portion of) the device or a plate. The unit and or display may be designed to be separate or detached (from the device), and therefore not distant or connected with a wire, or may be physically connected (such as integral). The display and/or unit may have a (e.g. metal) guard to prevent or mitigate (accidental) damage from impacts (hitting or striking).

The display may be able to show various desired and/or useful parameters, such as force, location (and/or accuracy). It may be able to display the number of impacts. It may comprise a timer so one can time the impacts (and relate them to location of impacts).

The device may be able to display a score (or other parameter) based on force and/or impact, suitably combined with accuracy and/or location. In particular, it may be able to give a force and (such as multiplied by) accuracy parameter.

The device may be capable of being horizontal, so as placed/; located on the floor. It may (capable of being) fixed to the floor to reduce, restrict or prevent (its) movement. Equally, it may be capable of being mounted vertically, such as on or to a wall. Alternatively, it may be capable of being positioned in an angled position, for example located between the feet of a user. It may be able to be carried and/or lifted by a human adult.

Suitably, the target area (which may comprise or be made of rubber) is not intended to be hit or impacted by any part of the human body, such as hands, feet or elbows. The target area will usually be made of a hard material or comprise a hard surface, that may prevent or discourage this. This is because the target area is intended to be hit with (and so withstand an impact of) a slam ball, (sledge)hammer or mace, and not a part of the human body. It is therefore suitably capable withstanding multiple sledgehammer impacts. The target may thus be impact resistant. It may have or comprise a hard material or hard surface, that is substantially non-deformable (for example, this will not be a mat or compressible material).

The device may therefore comprise multiple circular layers or rings, for example comprising nitrile rubber, polycarbonate, metal (such as steel). It may be portable such that it can be lifted (as a whole) by a person (such as a man) it may have a maximum weight of 25 kg, such as 30 kg, optimally up to 35 kg. It may weight at least 10 kg, 15 kg or 20 kg.

Suitably the device is substantially circular. Its diameter may vary, and can be any suitable size, but for example may be from approximately 40 or 50 cm, suitably up to 60 cm, but preferably around 45 cm. It may be (from) 20 cm, 30 cm or 40 cm high (or in depth). Suitably it is no higher than 40 cm, 50 cm or 60 cm.

The device may be (designed to be) able to withstand impacts of at least 2000N, 4000N (Newtons), 6000N, 8000N or even 10,000N.

Preferably, there are multiple sensors. This may be an (e.g. 8) layer design. The device is suitably robust and is adapted to absorb significant pressure.

The device may have a detachable top or surface layer which may be replaced, for example, after it is worn or after multiple impacts.

The generally circular layers may be held together with a central or telescopic rod. There may be one or more fasteners.

The device may be able to measure force or impact, for example of each hit. It may be able to measure and/or calculate cumulative force for multiple hits, for example for each user. It may also be able to measure the (location and thus) accuracy of each hit or strike.

It will suitably be able to measure power (in Watts or other suitable parameter or unit, which may be a standard or SI unit or another useful calculated score or points). The number of hits per minute (the device will have a clock or timer). It may therefore be able to have an algorithm that can measure power and/or accuracy (of the strike or impact).

In the invention, the device preferably can display parameters for each hit (or strike or impact). It may therefore display the force and/or impact of each hit, and the cumulative force of all of the users' hits. This can allow a users' hits to be measured and scored against others.

The device is suitably robust, sturdy and/or almost indestructible.

Suitably, it can measure force per hit, and provide a measurement of cumuli force. It may also have the ability to reset scores, for example for a new or different user.

Suitably, the device is still stable (does not move) even when struck and/or hit off-centre. It should be movable and/or portable by a human, and it may be adapted to be secured to the floor if necessary. It may be (self-)battery powered (and so may comprise a battery) and/or powered by mains electricity.

The device may provide an indication of accuracy of strike. For example, it may score 100% if hit in the bullseye, 75% in the second ring, 50% in the third. It may thus provide multiple score measurements, such as force, location(accuracy) and/or other parameters.

The device may therefore provide a way of measuring work or effort of a sledgehammer swing.

The user (or striker) may be using a sledgehammer, a mace, or a slam ball. The device comprises a target (or impact) area to allow aiming. It may have a bullseye target. The performance monitor may be integral and/or attached to the side. The top or target area surface may comprise a rubber (upper surface) and so may resemble hitting a rubber (for example, tractor) tyre. It should have some flexibility, and allow a small amount of bounce, but largely should be non-deformable.

The display is suitably simple, easy to use, has the ability to change units and/or the ability to change parameters. Preferably, it can be integrated into the side of the device or “stump”.

The device may have an integrated performance monitor or measuring equipment. It may comprise a clock, so it can suitably detect hits per minute, the last hit score, hit score accuracy, and/or accumulative score. It may be able to change units and/or change the display. The device may have Wi-Fi connectivity.

The device may comprise multiple layers, preferably all substantially circular and with the same or similar diameter. A top surface may comprise (e.g. nitrile rubber), and this may be the point or target of impact. There may then follow (below) one or more foam layer(s). There may also be also one or more polypropylene layers, polycarbonate layer(s), layer(s) of foam and/or stainless steel.

All of these layers may suitably be substantially circular and of similar diameter.

The sensors may be passive and/or active. Preferred (e.g. passive) sensors may comprise pho-electric sensors. Piezoelectricity is the charge created across certain materials when a mechanical stress is applied. A piezoceramic material (usually a crystal, and sometimes a ceramic) can be located between metal plates. When the piezo material is compressed, squeezed, or hit, it can generate electricity. These materials maybe comprised of crystals and/or ceramics.

Other sensors can be active, for example resisted FSR, capacitive or inductive electromagnetic.

Capacitive sensors use an electrostatic field (such as a small electric charge) and can provide a base reading. If objects get close, for example that conducts electricity, the electrostatic field is altered, and a controller can monitor this change and interpret it. These sensors can be relatively accurate and durable.

A resisted FSR may directly measure compressive force. It may comprise a couple of (e.g. thin) layers, suitably the top one being slightly flexible. When pressed, a top layer may touch a layer below, and so complete and electronic circuit. This can inform where and how much pressure has been applied.

Another type of suitable sensor is an inductive electromagnetic sensor. Conductive (aka electromagnetic or eddy current) sensor projects a magnetic field or (small electrical charge) and may give a base reading. If an object becomes near or in proximity, for example that is magnetic, the magnetic field is altered. A sensor or controller may monitor this change and interpret it.

The device may be able to measure and/or calculate (one or more of):
power×accuracy (FIGS. 4-6);
power×accuracy, such as using direct electrical force measurements (FIG. 2);
Is designed to give a single adjusted score based on the above (our FIG. 4-6);
Is designed to be comparable (all units give the same score) and/or repeatable (same units gives same score on hit 1 as hit 1,000,000) (FIG. 4);
Is designed to withstand a slamball, sledgehammer, mace and/or is a fitness unit (FIGS. 7 & 12); and/or
Is designed to be modular, for example to enable down-banger, mid banger, and low-banger hits, suitably as well as (elevated) (sledge)hammer (FIGS. 8 & 12).

Preferred features and characteristics of one aspect of the invention are equally applicable to another aspect mutatis mutandis.

### DESCRIPTION OF THE DRAWINGS

The invention will now be described by reference to the following drawings, which are not intended to be limiting:

FIGS. 1, 2 and 3 show four types of sensors that can be used in the device of the invention.

FIGS. 4, 5, 6 and 8 compare the old traditional tyre sledgehammer training (prior art) with the training device of the invention;

FIG. 7 shows a display that can be integral with the device of the invention;

FIGS. 11, 13, 14, 15 and 16 show exploded view of the components of the device of the invention;

FIG. 12 shows a number of photographs of the device of the invention;

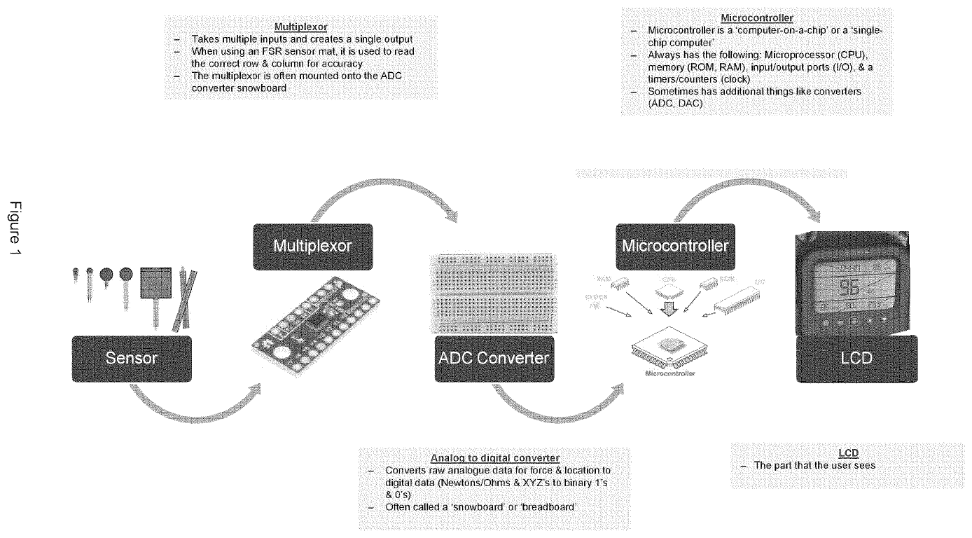

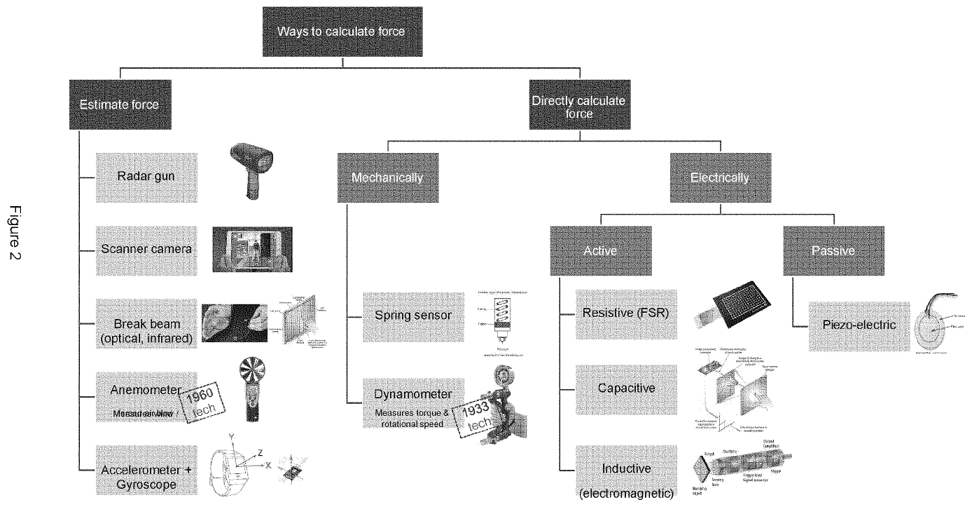

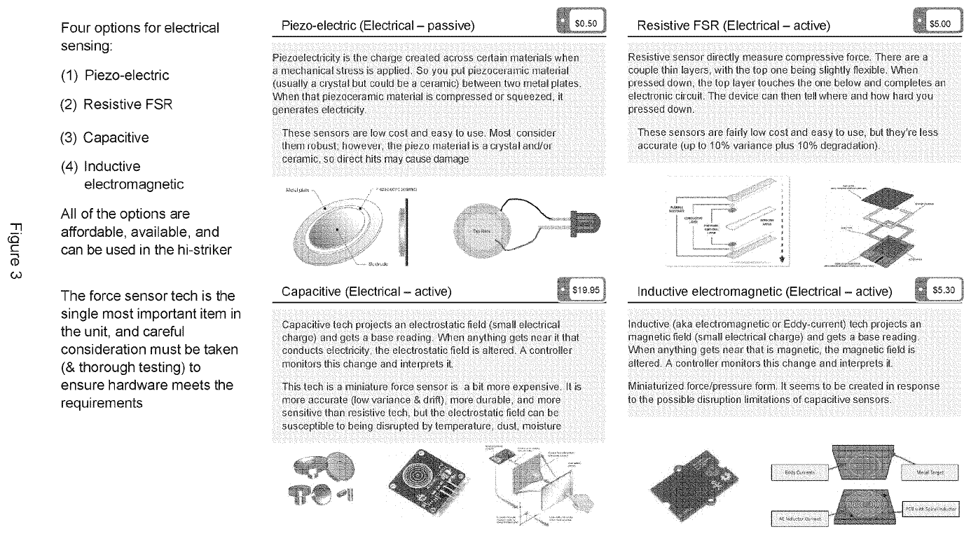

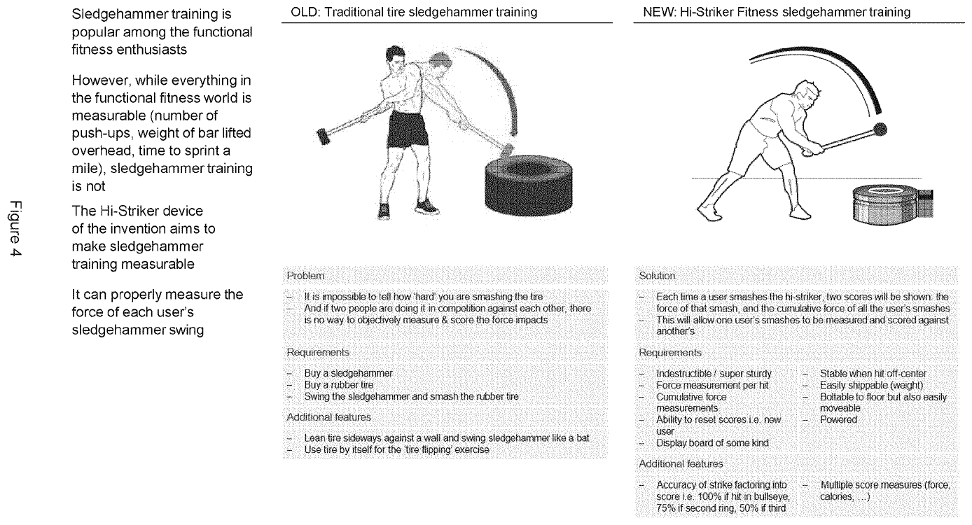

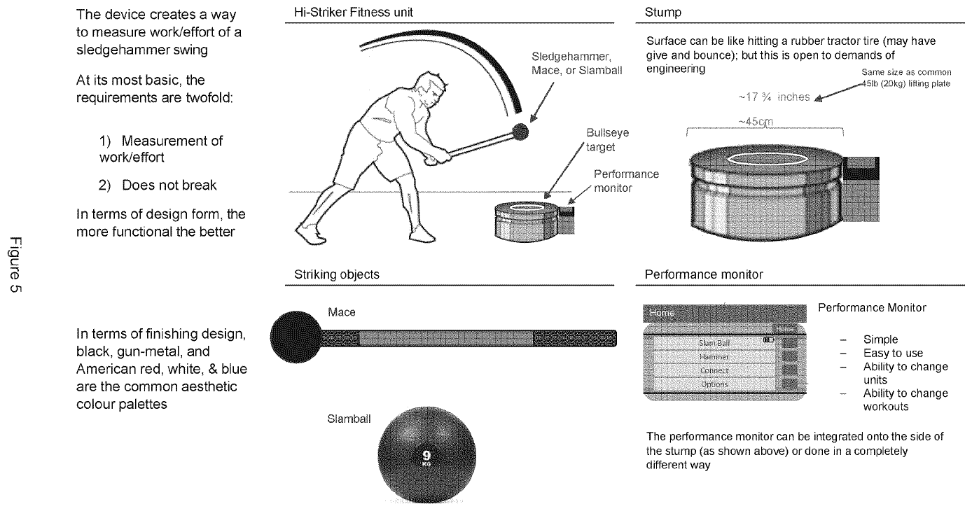

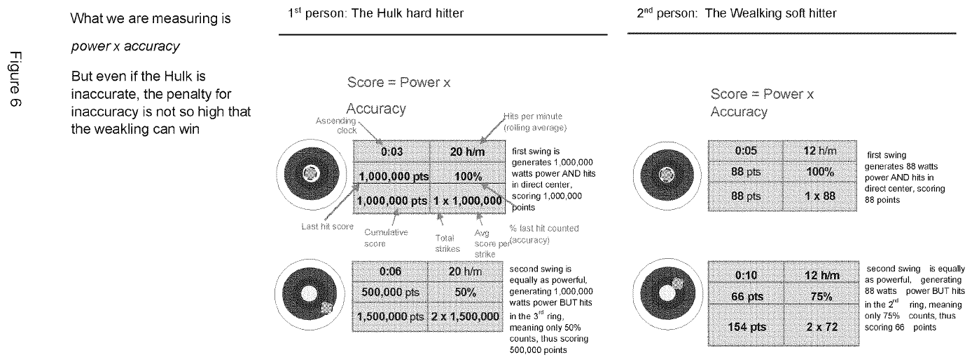

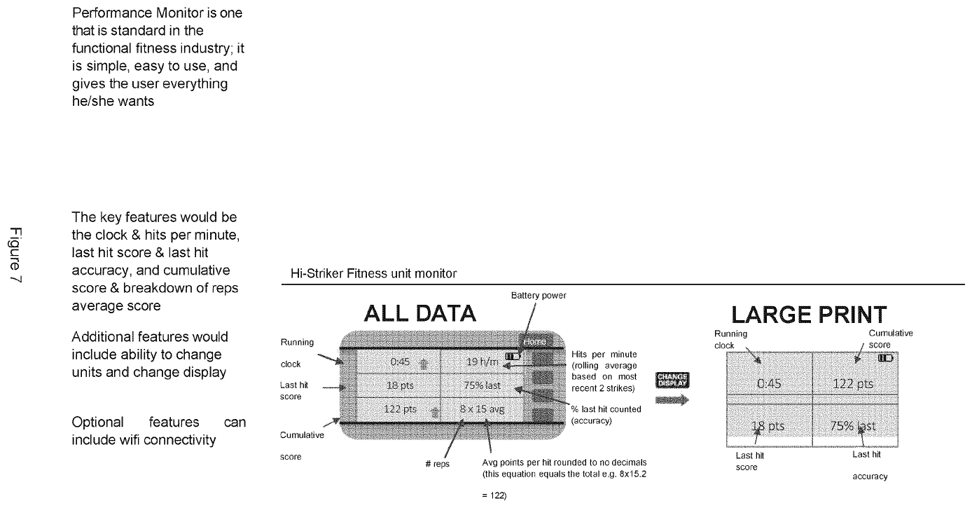

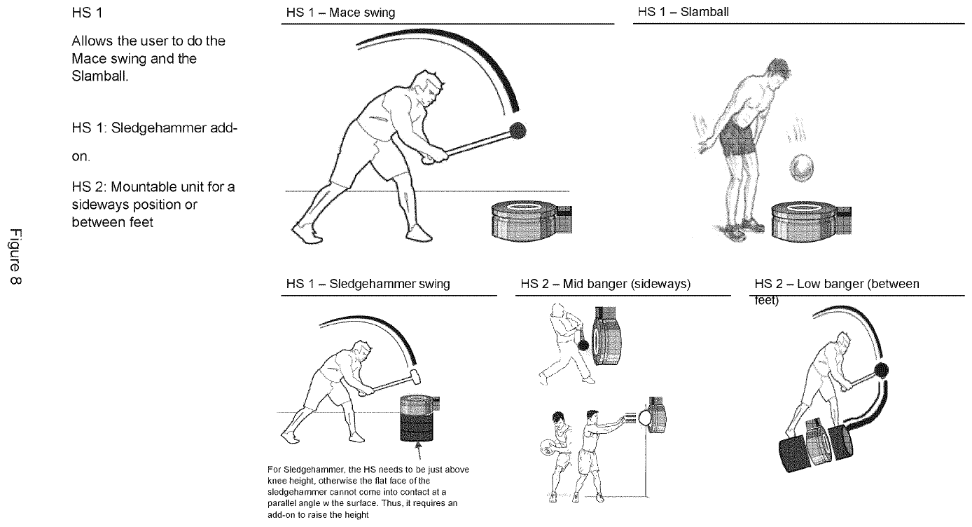

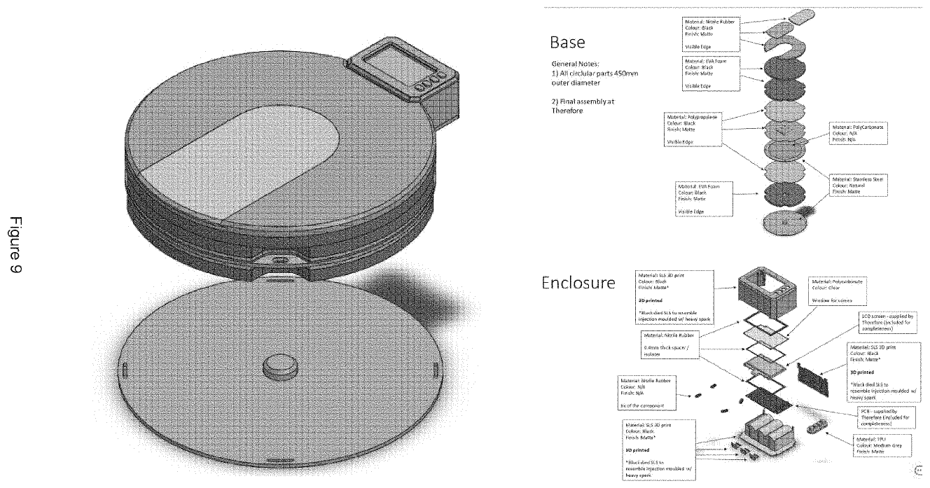

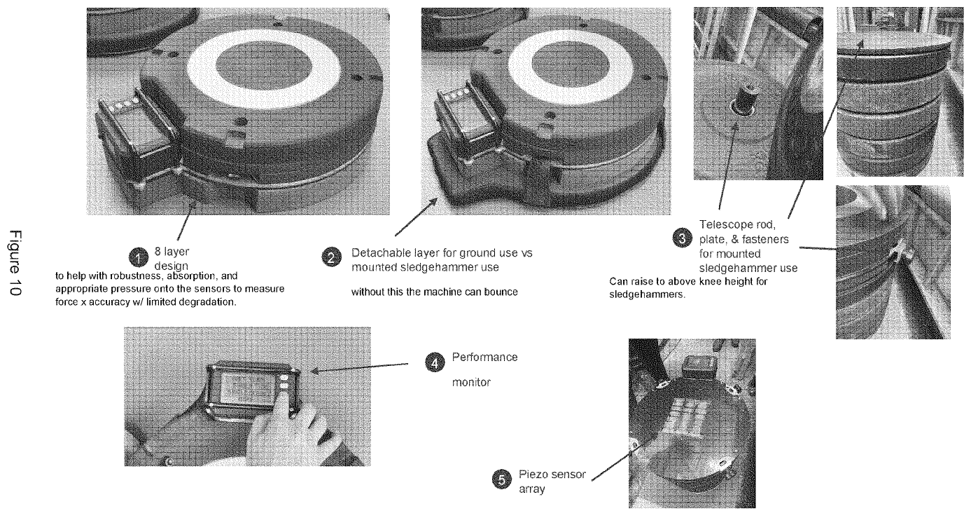

FIG. 7 is an exploded view of the major components of the device according to the invention.

The invention will now be described by reference to the following Examples, which are also not intended to be limiting.

### Example 1

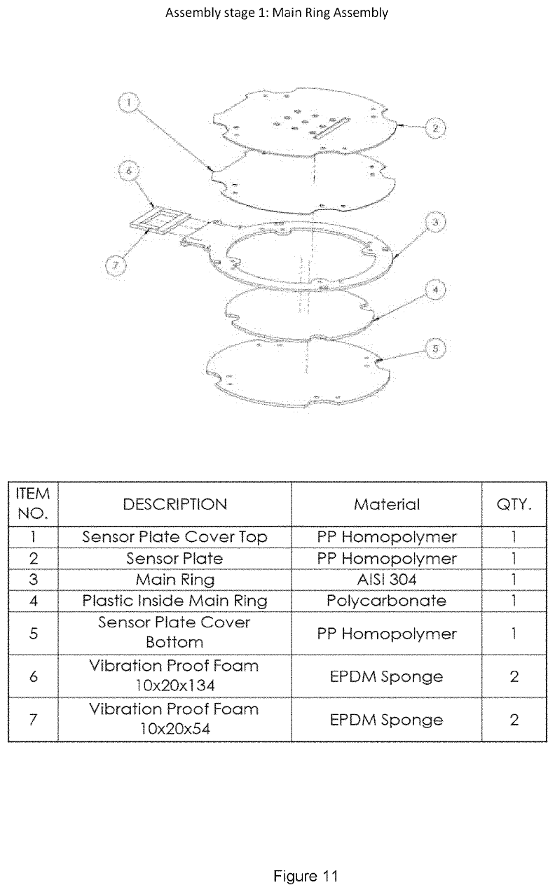

A force and impact measuring device of the invention is constructed as shown in FIG. 11. This is an exploded view and shows the layered plates used to construct the device and lists the materials each plate is made of.

The sensors detect the force and location of the impacts. The sensors can measure the force of the impacts. However, the accuracy % is calculated differently.
100% accurate: If the center sensor receives the most force and all the other 8 sensors around it receive an equal measure of force, then there is a 100% accuracy read-out.
75% accurate: If a side sensor receives equal force to the center sensor, one gets 75% accuracy score.
50% accurate: If the side sensor receives more force than the middle sensor, one gets a 50% accurate score.
25% accurate: If the side sensor receives force and the center one nothing, you are 25% accurate.

After the force and accuracy are measured, an algorithm can calculate them together to produce a final adjusted force score. One can adjust that total score based on the combination of sensor power and overall % accuracy. For example, the raw sensors say 100, then the initial score is 100. If the sensor impact is split evenly amongst the central ring (100% accurate) and middle ring (50%) accurate, then the accuracy adjustment factor is 75%. So a 100 raw score multiplied by 75% accuracy equals a final score of 75. If the accuracy was different, then the 100 raw score could be multiplied by 100% accuracy for a 100 score or 25% accuracy for a 25 score.

### Example 2

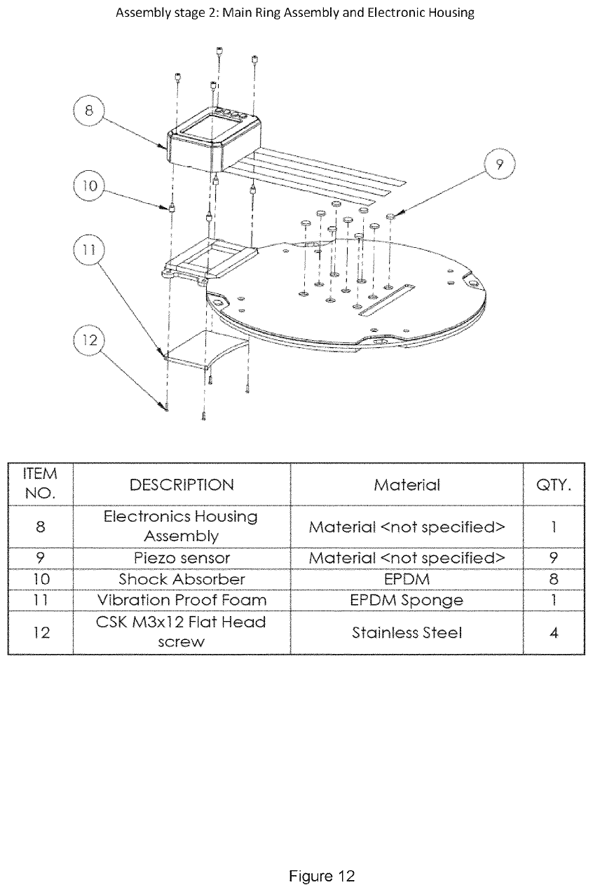

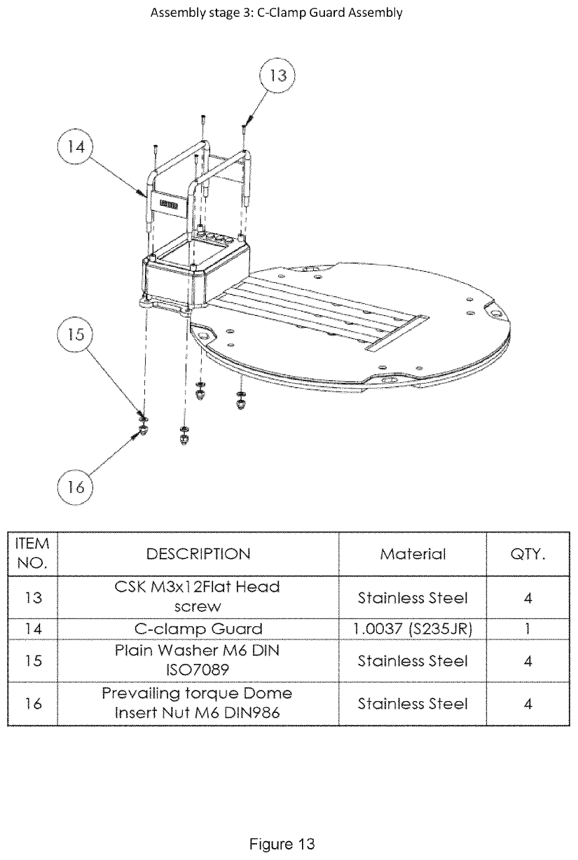

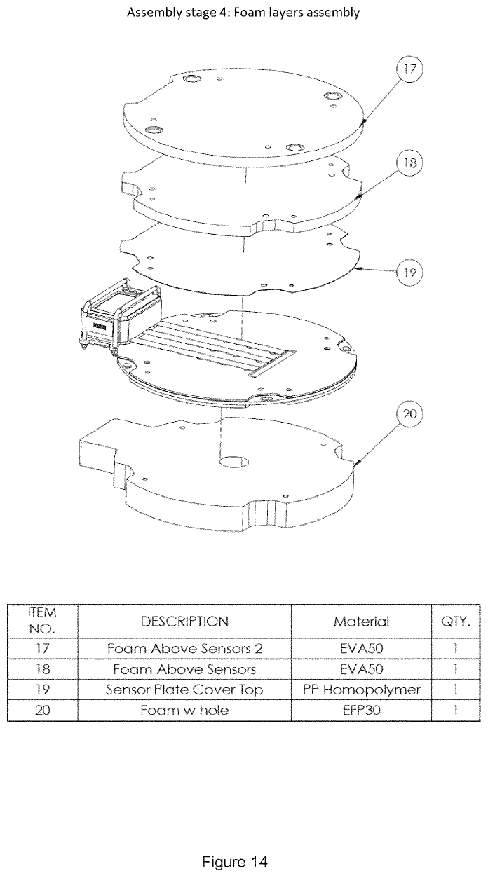

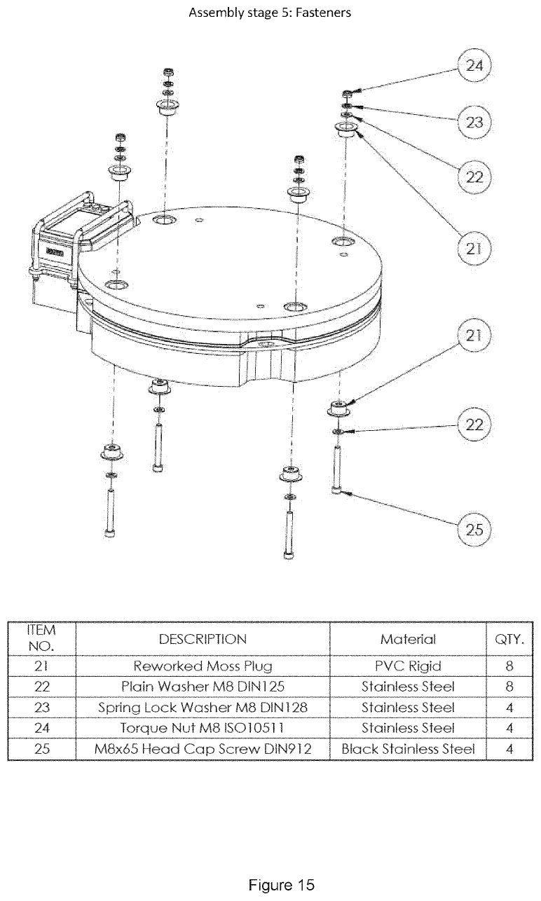

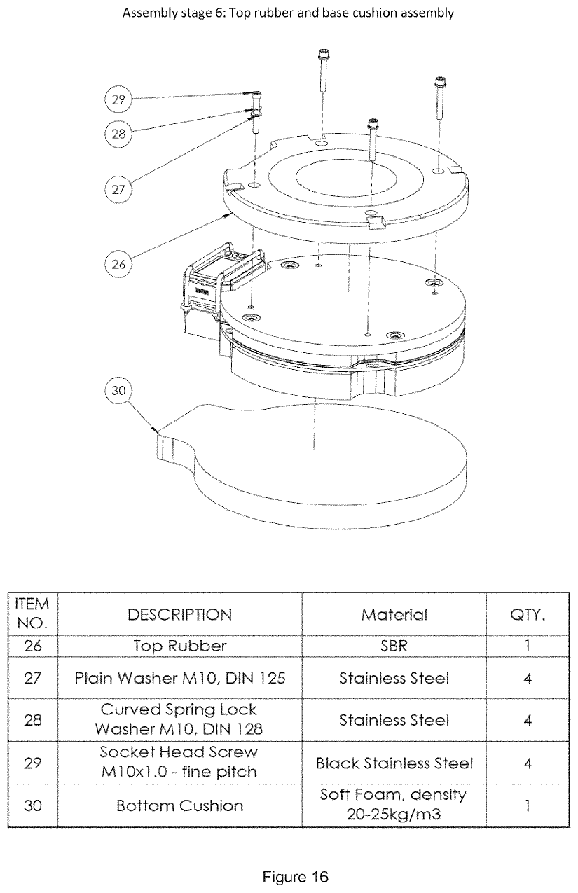

A second device of the invention is constructed using the diagram and instructions of FIGS. 12 to 18.

A different algorithm variation is used to provide a score to the user.

A tiered sensor grid is included. The center tier is x4 and the wide tier x1.

A different formula construct has the sensors measure an inherent accuracy adjusted force score and then show an accuracy score separately.
(a) The score is calculated on the sensor power with a boost to the center sensor. The middle sensor registers 20 and surrounding sensors 20. This gives a score of: 20×4+20=100.
(b) Accuracy is a separate measurement that is produced as an end-result to show on the display screen.

1. A force measuring device comprising a target or impact area, the device having one or more sensor(s) capable of measuring force and/or impact and the location (or accuracy) of an impact (or hit, or strike).

2. A device according to claim 1 which is:
(a) a high-striker, or a strength tester device or apparatus, such as an (electronic) test-of-strength or high striker (game) machine or apparatus, a fitness evaluation or capability device, and/or a strike assessment apparatus (suitably there is no bell, (moving or sliding) puck and/or vertical tower or slide);
(b) portable and/or can be (easily) moved or carried/lifted by a human; and/or
(c) is capable of being located horizontally (such as on the ground or floor) and/or vertically (such as mounted or located e.g. on a wall).

3. A device according to claim 1 which can measure the force or concentration and/or location (of a strike or impact) or location or accuracy of impact; and/or
comprises one or more sensor(s) able to measure said parameter electrically, electronically or digitally (but not mechanically).

4. A device according to claim 1 which has one or more force or impact sensor(s) and/or means for measuring or calculating force (of a strike or impact).

5. A device according to any preceding claim 1 which comprises one or more location or (accuracy) sensor(s), and/or one or more sensor arrays for detecting location of impact.

6. A device according to claim 1 which is substantially circular or cylindrical in shape and/or resembles a “stump”.

7. A device according to claim 1 wherein the target area is designed or capable of being impacted, hit or struck, such as by a (sledge) hammer, mace or slam ball on multiple occasions.

8. A device according to claim 1 wherein the target area has one or more (such as a series of) concentric rings, suitably where the rings are coloured (and/or differently coloured).

9. A device according to claim 8 which comprises one or more sensors for each ring.

10. A device according to claim 1 which comprises multiple layers and/or plates, suitably each being substantially circular and of a similar or same size or diameter.

11. A device according to claim 1 which comprises an integral display and/or is capable of displaying one or more impact parameters.

12. A device according to claim 1 which comprises a performance monitor.

13. A device according to claim 11 where the display is integral to the device, for example located on the side of the device (and is not separate or detached).

14. A device according to claim 1 which can provide an indication of accuracy and/or location of the strike or impact.

15. A device according to claim 1 which can measure and/or display the number of impacts, the time of impacts, and the location of impacts.

16. A device according to claim 1 which is not adapted to be impacted or hit by any part of the human body.

17. A device according to claim 1 wherein the target area comprises a hard material or hard surface that is suitably non-deformable.

18. A method of measuring and/or detecting force of an impact, the method measuring the force (or power or concentration) of an impact and as well as measuring location (such as accuracy) of said impact.

19. A method according to claim 18 wherein the device measures the force of an impact in a target area and measures the location (or accuracy) of the impact (or strike or hit) in a target area.

20. A method according to claim wherein one or more sensor(s) capable of measuring force and/or impact and the location (or accuracy) of an impact (or hit, or strike) or wherein the or each sensor(s) measure the or each parameter electrically, electronically or digitally (and not mechanically).

21. (canceled)
